(19) United States
(12) Patent Application Publication
(10) Pub. No.: US 2021/0158914 A1
(43) Pub. Date: May 27, 2021

(54) SELF-REFERENTIAL CLINICAL SUPPORT AND QUERY SYSTEM

(71) Applicant: Dashboard Systems LLC, Lakewood, NJ (US)

(72) Inventors: Christina Mechelle Heard, Benton, LA (US); Ricky A. Stockton, Benton, LA (US)

(73) Assignee: Dashboard Systems LLC

(21) Appl. No.: 16/950,621

(22) Filed: Nov. 17, 2020

Related U.S. Application Data
(60) Provisional application No. 62/938,652, filed on Nov. 21, 2019.

(51) Int. Cl. G16H 10/60; G16H 40/20; G16H 50/70; G16H 70/60; G16H 50/20; G16H 15/00; G16H 40/67; G06F 16/245; G06F 16/28; G06K 9/00

(52) U.S. Cl. CPC G16H 10/60; G16H 40/20; G16H 50/70; G16H 70/60; G16H 50/20; G06K 9/00483; G16H 40/67; G06F 16/245; G06F 16/285; G06K 9/00456; G16H 15/00

(57) ABSTRACT

A method of identifying and selecting a singular medical diagnosis to be entered into a medical record as a part of a structured systematic clinical documentation improvement program, utilizing a dataset compiled from industry standard databases. A system embodying the method provides a unique healthcare provider self-querying device capable of identifying a singularly unique medical diagnosis to the exclusion of others within a classification. The invention provides the ability to clinically support the diagnosis selection process at the point of care with customization based on location of service and episode of care type.

### PRIORITY

This application claims domestic benefit from pending application No. 62/938,652, filed Nov. 21, 2019, and incorporated by reference.

### FIELD OF INVENTION

The present invention relates to a computer-implemented method and systems for singularly and uniquely identifying a medical description within a subset of a classification system for entry into a medical record.

### BACKGROUND

Choosing the right medical diagnostic description to describe a patient encounter is easier said than done. Electronic data sets exist for listing diseases and disorders within various classification systems. These public data sets are typically indexed numerically or alphanumerically. These data sets are used for various reasons within the healthcare system.

The International Classification of Diseases-Clinical Modification 10th edition (ICD-10-CM) is a globally used diagnostic tool for epidemiology, health management and other clinical purposes. The ICD-10-CM is the required classification system healthcare providers use in reporting medical conditions established during a patient encounter. The ICD-10-CM currently contains over 68,000 unique records and requires 1,318 pages of a pdf file to display. It is nearly impossible for a single healthcare provider to memorize, recall, and effectively use the entire ICD-10-CM database during a normal day of examining patients. Each time a healthcare provider writes a medical diagnostic statement, it becomes translated into an ICD-10-CM alphanumeric code prior to transmitting to third parties, such as healthcare facilities or insurance providers.

The ICD-10-CM code set is not used at the point of care by healthcare providers to review the level of specificity in a contemplated diagnosis prior to entry in a medical record. One reason is the size of the ICD-10-CM code set is too large for an individual healthcare provider to know or reference at the point of diagnosis. Instead healthcare organizations employ specially trained nurses or medical coders to review charts and produce a provider documentation query. Provider documentation queries are designed to clarify a diagnosis that is already documented but not perceived to be documented to the highest degree of specificity. Electronic medical records attempt to solve this problem by having clinical documentation aid modules integrated within the system. However, a well-known disadvantage of these modules is that they tend to produce unnecessary documentation queries based on system analysis of clinical indicators. Electronic medical records enhanced with documentation aids do not produce an exhaustive list of options, instead the system logic generates a pre-defined non-exhaustive options list. Currently there are no methods of uniquely identifying an individual medical diagnosis from all others in a classification system in a manner that is practical and useful at the point of care. Under the current state of the art, it is reasonable to question the benefit of having such a detailed classification system if it cannot be used practically at the point of care. For ease of understanding the invention, it is important to understand how the ICD-10-CM classification relates to the Medicare Severity Diagnosis Related Groups classification system as described next.

Medicare Severity Diagnosis Related Groups (MS-DRGs) is a classification system used to predict resource utilization based on cost factors associated with a specific medical diagnosis. The classification system is categorized according to specific patient attributes and then grouped into 25 Major Diagnostic Categories. This classification scheme provides a means to relating the types of patients treated by a healthcare facility to the cost incurred by the healthcare facility. What are referred to as a Principal diagnosis and a Secondary diagnosis are determined by the translation of a documented medical diagnosis in the medical record. The Principal diagnosis determines the MS-DRG classified for a specific in combination with the severity of illness of any Secondary conditions (indicated as MCC and CC status). The diagnoses that constitute both are part of the ICD-10-CM classification system. As stated in the CMS published document Design and Development of the Diagnosis Related Groups “One of the major applications of the DRG is a means of communicating with the physician community, the patients in each DRG must be similar from a clinical perspective.” However, this classification system is entirely invisible to the physician community until after they have had their documentation translated. The ability to correlate real-time clinical care to the inter-relationship between these two classification systems would be beneficial to the physician community. This classification system is applicable to short-term and long-term acute care healthcare facilities. Statistically derived numerical data based on the assigned MS-DRGs representing resource utilization and length of stay indicators are published annually and are representative of each specific MS-DRG as it pertains to each setting individually.

Discriminating between all the various description options for a condition as common as hypertension can turn into an arduous task for the healthcare provider. For example, the specificity required for completely describing a fracture in a patient's medical record requires the diagnosis to include several of the following details, and document these details in the patient's record:
1. Type—Open, closed, pathological, neoplastic disease, stress
2. Pattern—Comminuted, oblique, segmental, spiral, transverse
3. Encounter of care—Initial, subsequent, sequelae
4. Healing status, if subsequent encounter—Normal, delayed, nonunion, malunion
5. Location—Shaft, head, neck, distal, proximal, styloid
6. Displacement—Displaced, non-displaced
7. Classification—Gustilo-Anderson, Salter-Harris
8. Any complications, whether acute or delayed—Direct result of trauma
9. External cause—How did it happen and where did it occur

Most healthcare providers have difficulty independently documenting clearly, specifically, concisely and completely these details to the standard required by the ICD-10-CM. The degree of granularity in the ICD-10-CM standards is necessary in order to track medical complications and prevalence of certain conditions. The information obtained from the use of coded medical descriptions is also used to estimate the cost of treating specific medical diagnoses which in turn is a factor in setting reimbursement rates from third-party payor sources.

In response to the lack of granularity reported in medical data, most healthcare providers use a combination of provider education, electronic medical records (EMR) documentation assistant modules, and clinical documentation improvement programs.

Provider education typically consists of certified medical coders or clinical documentation improvement specialists conducting physician in-service training. The training is usually limited in scope and directed toward high risk documentation deficiencies. It is common for healthcare facilities to hire outside consultants to provide specialty-specific medical documentation education to their credentialed healthcare providers. Provider education can produce improvements in physician documentation, but due to the large volume of medical descriptions contained within the whole ICD-CM-10 code set, the effectiveness of such education is extremely limited.

Electronic medical records systems often include documentation assistant modules. These modules work by using a combination of structured or unstructured databases in addition to text searches to generate electronic queries. The provider initially enters the demographic data for a specific patient and establishes a working diagnosis. Based on provider's input, an options list of alternative medical descriptions is displayed for the providers consideration. Most EMR documentation models synthesize diagnostic data such as patient specific lab results, and generate additional queries based on how the branching logic within the system is designed. Patient-specific clinically derived information serves as the source for the specific branching logic used in the documentation assistant modules.

Electronic medical records equipped with physician documentation support tools require a multistep process. Depending on the search terms used, the provider may be required to answer additional questions prior to being presented with a non-exhaustive list of options for selection. In addition, the analysis of clinically derived indicators can produce queries that may not be relevant to the patient encounter. Electronic medical records equipped with documentation support tools can serve as an aid to improve physician documentation, but they only produce a range of selection options and are known to pose unnecessary questions that healthcare providers are then required to address.

Clinical documentation improvement programs are designed to implement strategies within an organization to ensure that provider documentation is accurately reflective of the patient's clinical condition. The process usually requires a specially trained registered nurse or certified medical coder to conduct extensive chart reviews. The chart reviews primarily consist of identifying clinical indicators that suggest that a previously documented medical condition could potentially be further specified. This process entails the formulation of a manual query. Manual queries are placed on the chart and flagged for the healthcare provider to address. Those that work in the clinical documentation improvement field also identify areas for further improvement and devise additional tools such as reminder templates. According to the Bureau of Labor Statistics this industry's growth rate is faster than the national average for all occupations. However, this process is also known to result in the healthcare provider having to take time to respond to unnecessary questions concerning a clinical indicator. In addition, when a manual query is drafted during this process, the options list presented to the healthcare provider is a non-exhaustive list, determined by an individual with less clinical knowledge than the healthcare provider responsible for establishing the diagnosis.

Improving physician documentation and evaluating the effectiveness of clinical documentation improvement programs have been extensively researched in the medical literature. In evaluating the effectiveness of clinical documentation improvement programs, all research conducted on the topic to date concludes that much more improvement is needed. According to medical researchers and advice posted on the official ICD-10-CM website, improvement in physician documentation can only be achieved by deploying more human capital to review medical records, spending more money on healthcare provider education, and investing in advanced technological solutions that can better predict the significance of clinical indicators. The inventors of the Self-Referential Clinical Support and Query System disagree with the current recommendations published by medical researchers and the governing bodies responsible for maintaining the ICD-10-CM code set. Instead, the inventors have discovered and developed an unconventional solution to a problem that has been plaguing the healthcare industry for decades.

The conventional methods being used in the current state of the art solutions to increase the granularity of reported medical diagnoses are designed and implemented based on the following assumptions: (1) The ICD-10-CM code set is too large, therefore an exhaustive list of medical conditions would not be practical to produce for a physician's review prior to completing the medical record; (2) Analysis of clinically derived information is needed to automatically generate documentation improvement queries; (3) Trained nurses and or medical coders are needed to review charts in order to identify medical conditions that are documented but are just not documented to the highest level of specificity; and (4) Physicians and other qualified healthcare providers need ongoing education to improve their knowledge base regarding how to specify certain medical conditions.

Existing electronic and manual healthcare provider queries do not include the universe of medical descriptions containing medical diagnoses as part of the selection options from which the healthcare provider is expected to choose. Instead conventional methods use a subset of options based on a previously determined interpretation of clinical indicators, which clinical indicators may or may not be relevant to the actual patient under examination. Generating queries based on clinical indicators is a known source of frustration for practicing healthcare providers and is well documented in the literature as contributing to low provider morale across all types of healthcare settings.

In the context of a busy healthcare provider's daily routine, checking each contemplated or established medical diagnosis by conventional methods would require additional personnel to manually look up each contemplated diagnosis occurring in each patient encounter. The additional personnel would have to cross reference each description against all possible descriptions containing the queried medical diagnosis from all descriptions where the medical diagnosis appears in the description title of the code.

The present invention addresses each assumption in the following manner and in a method embodied below producing a novel method for physicians to “self-query” their own contemplated or previously written medical diagnosis which would then be entered into the patient's paper or electronic medical record. A computer-aided process has the potential to vastly increase the granularity and/or specificity of reported medical diagnoses. This solution uses a systematic approach where each medical condition diagnosed can efficiently be self-checked against all possible medical descriptions within the ICD-10-CM code set, using the same medical diagnosis described in the ICD-10-CM alphanumeric code description. This computer-assisted process is a cost-effective solution, potentially reducing the labor expense associated with manual chart reviews as currently occurring in the medical industry. This computer-assisted process also has the potential to optimize existing electronic health records by having the healthcare provider enter a more specified version of a medical diagnosis from the start, reducing the need for later analysis or queries. A more specified description entered from the start is likely to lead higher levels of granularity in other documents contained within the electronic medical record, and improved patient treatment.

The computer-assisted process combines data elements with different values applicable to the type of healthcare facility simultaneously displayed on a user-interface eliminating the need for separate site-specific solutions. The ability to simultaneously display different values of data elements linked to the same medical description is a time-saver for a physician that practices routinely in both settings.

The inventive solution addresses assumption 1, that the ICD-10-CM code set is too large, and therefore an exhaustive list of medical conditions would not be practical to produce for a physician's review prior to transmitting to third parties. The ICD-10-CM code set contains over 68,000 unique alphanumeric codes, but does not contain 68,000 unique medical descriptions associated with the alphanumeric codes. By discovering that the medical descriptions can be abstracted from each alphanumeric code, subsets of the entire classification data set were produced. Once isolated, each medical description was rejoined with an alphanumeric code associated with that description based on episode of care and encounter type. The additional data elements were then mapped to each description within the modified ICD-10-CM classification. The ICD-10-CM modified version was then arranged in the database in a specific order that would result in the individual descriptions visible on the user-interface in a head to toe descending sequence. This process resulted in the total number of applicable medical descriptions being reduced by approximately 48 percent for each query posed when the data set was normalized to reflect the pre-selected location of services and episodes of care. Despite the reduction in the number of records, the options produced via text search include the universe of applicable diagnosis descriptions from the original classification system for the physicians' review.

The inventive solution addresses assumption 2, that analysis of clinically derived information is needed to automatically generate documentation improvement queries. The inventive and structured process should greatly reduce the number of automatically generated queries. At the point of diagnosis, the healthcare provider now has a chance to review, select and transcribe word for word the most clinically applicable description into the electronic medical record. This has the potential to optimize the electronic medical record system by decreasing the resources needed to generate the automatic query in the first place, and to improve the granularity of medical information derived from diagnosed conditions in an existing EMR.

The inventive solution addresses assumption 3, that trained nurses and/or medical coders are needed to review charts in order to identify medical conditions that are documented but are just not documented to the highest level of specificity. A “self-query” process implemented by the healthcare provider follows a defined series of steps which should greatly reduce the number of manual queries that need to be attempted. A more accurate and specific diagnosis reduces the need for later review.

The inventive solution addresses assumption 4, that physicians and other qualified healthcare providers need ongoing education to improve their knowledge regarding how to designate certain medical conditions. Ongoing education is always recommended; however, the successful use of the present invention does not require the physician to have any advanced knowledge in terms of the specificity associated with a medical condition. The present invention is designed to work by having the physician or other qualified healthcare provider enter less specific terms associated with a medical condition and then review a list of results containing those terms from the displayed options of a single user-interface screen.

### SUMMARY

A method of identifying and selecting a singular medical diagnosis to be entered into a medical record as a part of a structured systematic clinical documentation improvement program. The method includes the following steps. First, the healthcare provider enters a text search based on an unspecified version of a contemplated or previously written medical diagnosis into a user-interface. The system returns several options as results for the healthcare provider to review and compares with the actual patient conditions. The healthcare provider then selects the description that best represents the clinical condition reviewed and enters the selected description to a paper or electronic medical record. The process should be repeated for each medical diagnosis under consideration during a patient encounter. The method is preferably optimized for the type of healthcare facility where the patient is located.

Embodiments of this invention are directed to methods for positively identifying a singular medical description by entering the name of a medical diagnosis to be uniquely identified within a classification to the exclusion of others within the classification. At least two different unique sources of data that contain data pertaining to the medical classification are provided as well as access to at least one individual searchable source. Data from these two different and unique sources of data are compared against each other based on text entry of a medical diagnosis to provide a collection of preliminary options. Preliminary options are displayed on a single screen user-interface for review by a healthcare provider looking for a match, which match is then verified and validated. This potential match is then reconciled with data from at least one of the different and unique data sources, locating at least a portion of a medical description that may be associated with the medical diagnoses that is to be uniquely identified. The potential match and the portion of the unique medical diagnosis are then compared with other data records within the individual search data source, locating a complete medical description that may be associated with the medical diagnosis that is to be uniquely identified. It is then determined whether the complete unique medical description uniquely identifies the individual medical diagnoses to the exclusion of all others within the classification. The healthcare provider then enters the uniquely identified medical description word for word into a medical record. The healthcare provider performs this method of comparison for each medical diagnosis contemplated or written during a patient encounter.

The method provides clinical support for the healthcare provider at the point of care, with diagnosis-specific classifications customized to location of service and episode of care type. Optional features of the present invention include enablement of notifications via push messaging when significant changes occur in recommended clinical guidelines associated with any specific medical diagnosis within a classification of medical descriptions.

A system embodying the method provides a self-querying device capable of identifying for a healthcare provider a medical diagnosis to the exclusion of others within a diagnostic classification.

A key advantage of the disclosed method and system is that it allows a single healthcare provider to efficiently prepare a patient medical record without the current requirement of subsequent data validation by staff.

This unique computer-implemented system and structured non-conventional method is superior to all other conventional methods in the field of clinical documentation improvement by increasing the accuracy, granularity and specificity associated with reportable data containing a medical description. In addition, this system does not rely on clinical indicators linked to a specific patient encounter, instead assumes the healthcare provider understands the significance of clinical indicators associated with a patient encounter, thus avoiding the need to interrupt an otherwise busy healthcare provider.

### DETAILED DESCRIPTION

The preferred embodiment of the system and method disclosed herein utilizes a database which has been previously established from the known ICD-10-CM and MS-DRG data sets. The database is converted from a many to many data structure to a one to one data structure, where the data from the two data sets is optimized for use by the system and method disclosed herein. In particular, records from the data sets are preferably reviewed to remove records which do not relate to initial diagnosis situations or are not primary diagnosis classification records. The optimization of the database further sorts the data in classification code order.

One or more embodiments of the invention are directed to methods, apparatus and systems for singularly and uniquely identifying a medical diagnosis within a given classification to the exclusion of all other medical diagnoses within such a classification. An identification of a selected medical diagnosis, or group of medical diagnoses, within a classification or data set of medical diagnoses is electronically narrowed down to the actual, most clinically representative medical diagnoses to the exclusion of all others within such classification or data set of medical diagnoses.

The disclosed method preferably follows the process flow set forth below; however, it should be understood that other variations in process flows may be implemented for carrying out the method.

As a precursor to the disclosed method, the original large databases are manually scrubbed, namely, a “data reduction and mapping” operation is performed to produce manually compiled datasets. In one embodiment of the invention, the scrubbing enhances the compiled dataset by reducing the number of records based on the user-selected options. Such user-selected options may include episode of care type, or location of service. Preferably, these options are displayed for the user's review. The scrubbing produces only medical diagnosis relevant to the user-selected options, greatly reducing the number of records for verification and validation procedures that are performed in accordance with this invention.

The location of service used to create the reduced subset may refer to the typical length of stay at a facility such as long-term or short-term patient stays, and/or to the type of care offered at a facility, including without limitation hospital, out-patient clinic, in-patient rehabilitation, out-patient rehabilitation, nursing home, long-term care facility, or in-patient psychiatric facility.

Preferably, the compiled dataset used by a particular user is an established subset of all possible diagnoses. For example, a healthcare provider at a short-term acute care hospital would use a dataset that excludes long-term care diagnoses. Using a subset dataset, the system can be optimized for faster and more relevant processing, excluding diagnoses which may not be relevant to the location or facility type. Starting with a subset dataset, the verification and validation steps are optimized for faster output and a reduced number of records for review.

The compiled dataset preferably contains multiple records of multiple data fields, where the data fields include informational elements that provide context to the medical descriptions or other medical information. The specific data set files are accessible from a standard database, where the complete file or individual records can be customized based on desired need. Examples of fields preferably include, but are not limited to:
ICD-10-CM alphanumeric codes—A classification systems from the World Health Organization that has been transformed into a clinically modified version. The present version is the 10th edition with over 68,000 unique alphanumeric codes. In the United States, ICD-10-CM alphanumeric codes are required to be assigned to all medical conditions being submitted for reimbursement to third party payors.
ICD-10-CM diagnostic descriptions—The medical description used to describe the alphanumeric codes from the ICD-10-CM code set.
Major Complications and Co-morbidity Status—Useful in representing the acuity level that a specific medical description is associated with. This status is pre-determined by CMS and symbolized in the area of the user-interface containing the medical description it is assigned. Example: MCC yes or No.
MS-DRG—Medicare Severity-Diagnostic Related Group—Groups of medical diagnoses defined by the Centers for Medicare and Medicaid Services (CMS) as requiring similar utilization of resources within the Major Diagnostic Category groupings. Each MS-DRG has a relative weight assigned by CMS which can be correlated to reimbursement and utilization data from CMS. Example: MS-DRG 51
Long-Term Acute Care Relative Weight—A numerical value assigned by CMS to indicate resource utilization and applicable to licensed acute care hospitals with a CMS designation as being a long-term acute care hospital. Example: 1.15.
Short-Term Acute Care Relative Weight—A numerical value assigned by CMS to indicate resource utilization and applicable to licensed acute care hospitals. Example: 3.5.
Long-Term Acute Care Discharge Minimum—Represents 5/6 plus 1 day of the geometric length of stay associated with a particular MS-DRG assignment. This is used in determining patient progress. Example: 19.5.
MS-DRG Number—A number assigned to a specific medical description indicating a specific category of diagnosis groups based on the Medicare Severity-Diagnosis Related Group classification system. This is useful in comparing other medical descriptions mapped into the same category as a medical description under clinical consideration. Example: MS-DRG 51.
MS-DRG Title—A description assigned to all medical descriptions grouped into a specific category representative of the Medicare-Severity Diagnosis Related Group number. Example: Spinal Disorders and Injuries.
User Initiated Selection Process—The user must define which data set is displayed based on their needs by selecting between a plurality of options. The options represent levels of care or locations of service and the type of encounter. Encounter types may include initial, subsequent and sequalae. Location of services include acute care, long-term acute care, inpatient rehabilitation, skilled nursing facilities.

Each medical description and the corresponding data elements must be interpreted from the record and field for which it is found based on user-selected options. This selection process determines which file from the database displayed. For example, the user-selected option could have the value “Inpatient setting- long-term acute care/Initial encounter”. The later selection process would only display those records with a medical description representative of an initial episode of care occurring in a long-term acute care hospital.

In another example, the user-selected option could have the value “Inpatient setting- long-term & short-term acute care/Initial encounter.” The later selection process would only display those records with a medical description representative of an initial episode of care occurring in both long-term and short-term acute care hospitals. This feature represents improvement over prior art, as it would allow a healthcare provider who rounds daily at short-term and long-term acute care hospitals to consider data element values applicable to both settings and data element values unique to each setting from the same user-interface. This adds a new level of convenience and efficiency by combining locations of services.

A healthcare provider preferably initiates the method by entering one or both of a location of service and an episode of care type as user-selected options. As discussed, these factors are used to determine relevant subsets of the whole dataset which is comprised of all medical diagnoses or medical descriptions and associated diagnostic codes. The relevant subsets are applicable only to the factors provided, and preferably both factors are provided to result in the most relevant subset for later processing. Where the method is implemented on a dedicated device at a particular healthcare facility, the location of service and episode of care types may be pre-defined user-selected options, so entry may not be required.

The healthcare provider next enters patient condition data relating to an individually specific medical diagnosis (or medical diagnoses). This patient condition data is used to electronically locate and/or identify a medical diagnosis from within the relevant subset.

The entered patient condition data is processed validate and verify the incoming data by making sure that all input data fields contain at least one meaningful value, for instance, making sure that the medical description terms contain at least one recognized medical term. These validation and verification processes are performed to prevent any erroneous data from further processing.

The entered patient condition data is then compared with the compiled dataset, seeking potential matches for the specific medical diagnosis being searched for (i.e., the uniquely singular medical diagnostic description identified from a given modified classification or large dataset). The dataset may be a complete classification (the ICD-10-CM code set) a sub classification (ICD-10-CM modified short-term acute) or even a subset of a sub classification system (ICD-10-CM modified short-term-acute-subsequent encounter) or a combination of subclassifications (ICD-10-CM modified short-term acute and long-term acute-subsequent encounter). The potential matches may include enhanced data alphanumeric fields, enhanced data medical description fields, enhanced data MS-DRG number fields, enhanced data MS-DRG title fields, enhanced data resource utilization fields, enhanced data severity of illness fields (MCC status), and enhanced data length of stay indicator fields as well as other desired fields from the dataset.

Results from the initial search, a subset of medical diagnostic descriptions retrieved from the compiled dataset, are displayed for user interpretation and selection of the most clinically specific medical diagnosis. The retrieved information preferably includes a list of records containing the medical diagnosis related to the patient condition data, as well as corresponding alphanumeric ICD-10-CM codes, statistically derived data, severity of illness designation (MCC), MS-DRG number, and MS-DRG titles associated with each record.

As an optional feature, each medical diagnostic description displayed on the interface may include hyperlinks to various additional information sources available for the healthcare provider's access, if additional clinical support is needed at the point of care. The hyperlinked elements are preferably customized to each displayed medical diagnostic description. The following are examples of additional information sources.
1. ICD-10-CM alphanumeric codes—useful in identifying the disease or disorder category that the medical description derives
2. Medical Diagnostic Terms—Representative of stem word statements used as a keyword (such as the most elementary means of describing a medical diagnosis) search.
3. Correlated Major Complications—Representative of other conditions identified through medical research as being strongly associated with the medical description option associated with the hyperlinked record.
4. Diagnostic Tests and Diagnostic Indicators—Representative of standard diagnostic tests and values associated with the medical description option of the hyperlinked record.
5. Associated Pharmaceuticals—Representative of current medical recommendations regarding drug treatments associated with the medical description option of the hyperlinked record.
6. Associated Articles, Journals and Other Media—Representative of current medical research or other medically oriented information associated with the medical description option of the hyperlinked record.
7. Interdisciplinary Treatment Plans—Representative of current recommendations care plans that may include but not limited to medical, nursing, respiratory therapy, physical and occupational therapy treatment plans applicable to the medical description associated with the hyperlinked record.
8. Environment of Care and Infection Control Measures—Lists the types of evidenced based recommendations need to be implemented in the physical plant and for infection control purposes that are need for the medical description associated with the hyperlinked record.
9. Visual depiction of Medical Condition—If applicable a visual image associated with the medical description (e.g. skin tumors, images of the types of bone fractures) are displayed for user reference and associated with the hyperlinked record.

It should be appreciated and understood that the customization of the hyperlinked records occurs to the extent applicable to location of services and episode of care types. For instance, the treatment plan for a bone fracture is significantly different in an emergency room for the first time treatment versus in a rehabilitation setting, where a subsequent encounter would require an entirely different reference source for clinical consideration. This customization occurs automatically with the user-selected options indicating location of service(s) and episode of care type(s) prior to key word search of a medical description.

The healthcare provider selects the most clinically relevant medical diagnosis presented in the options list. The healthcare provider may then select one or more of the retrieved records for entry into the patient's medical record. As the retrieved records contain the proper ICD-10-CM codes for the patient diagnosis, using the disclosed system and method improve accuracy of patient medical records and reduce processing time and errors introduced by subsequent handling of the patient medical record.

The system provides a reconciliation process to validate accuracy of reporting the entered medical diagnosis prior to translating to third parties. The process includes printing the retrieved options into a physical report. The report is then used by the healthcare provider and/or staff to reconcile the entered medical diagnosis with other options in the classification that was searched.

Optional capabilities of the system include the ability to edit, duplicate, export, copy, and save offline the retrieved records, and enable notifications, among others. The enablement of notifications may include the ability to automatically generate a push notification message indicating that a significant change has occurred in the recommended guidelines or recommendations associated with a specific medical diagnosis. For instance, the recommendations regarding infection control measures and personal protective equipment and other physical plant guidelines have changed frequently since the evolution of the novel virus Covid-19. The capability to send up to the date notifications, would expediate dissemination of this type of important public health information.

A system performing the method may be implemented on the personal mobile device of a healthcare provider, or at a workstation at the healthcare facility. The device preferably includes a display, data entry interface, data storage, a processor, and communication with a cloud-based server, progressive web site, Rest API, or on-site server. The data entry interface may communicate with a text search engine. The processor may communicate with a report generator.

A system performing the method preferably includes a client device and a database processing subsystem, as are well known in the art. The client device may be any known computing device that allows a user to input and receive information or data. A user may input patient condition data relating to a medical description at the client device, which patient condition data is compared by the database processing subsystem with the compiled dataset. The client device preferably includes a display screen for viewing the results and any reports. The database processing subsystem preferably hosts or is in electronic communication the compiled dataset.

The system further may includes a user-defined selector and workflow management module and/or an operational report generator, for supporting the operation and administration of the various embodiments of the invention, such as controlling the management and monitoring of the batch progression of the invention. These functions may be accomplished using known management tools (e.g. Zoho) or any other relational data stream management system.

The user-defined workflow and operational report generator may generate standard reports and operational reports for use by the healthcare provider, healthcare facility and administrative staff, using known report and query tools. As described in more detail below, the standard reports may include but are not limited to, file reports (e.g., modified versions of international classification of diseases, modified versions of the MS-DRGs classification system), data validation and verification reports, reconciliation listing reports, individual search healthcare provider reports, and the like. Operational reports include those reports that used by the system and/or by users of the system to track and manage data loads, refresh cycle executions, execute the individualization procedures, as well as other systems operations in accordance with the various embodiments of the invention. For instance, operational reports may include, but are not limited to, data load status and statistics reports, refresh cycle execution reports, web service interface operation reports, medical description individualization execution results and statistics reports, system error listing reports, and the like.

The various embodiments of the invention may be embodied as a computer data-set program product stored on a program storage device. These program storage devices may be devised, made and used as a component of a machine, utilizing electronics with or without built in operating system databases to electronically perform the method steps of one or more embodiments of the invention. Program storage devices include but are not limited to mobile phones, tablets, workstation computers. A computer readable program code means in known source code may be employed to convert the methods described below for use on a computer. In one or more embodiments, the computer programs or software incorporating the process steps and instructions described further below may be stored in any conventional computer or portable device.

While certain novel features of the present invention have been shown and described, it will be understood that various omissions, substitutions and changes in the forms and details of the device illustrated and in its operation can be made by those skilled in the art without departing from the spirit of the invention.

We claim:

1. A method of improving the accuracy of a medical diagnosis of a patient by a healthcare provider, where the healthcare provider has examined the patient and identified at least one patient condition, the method comprising the steps of:
receiving patient condition information from the healthcare provider, the patient condition information comprising a first descriptive term;
comparing the first descriptive term of the patient condition information with an existing dataset of diagnosis classifications, and deriving a relevant subset of diagnosis classifications based on relevant matches with the first descriptive term;
displaying the relevant subset of diagnosis classifications for the healthcare provider to review and select an item from the relevant subset of diagnosis classifications for entry into a patient medical record.

2. The method of claim 1, further comprising:
receiving facility information about a healthcare facility where the patient is located;
comparing the facility information and the patient condition information with the existing dataset of diagnosis classifications to derive the relevant subset of diagnosis classifications.

3. The method of claim 1, further comprising:
receiving a second patient condition information, the second patient condition information comprising a second descriptive term;
comparing the second descriptive term of the patient condition information with the existing dataset of diagnosis classifications, and deriving a second relevant subset of diagnosis classifications;
displaying the second relevant subset of diagnosis classifications for the healthcare provider to review and select an item from the relevant subset of diagnosis classifications for entry into a patient medical record.

4. The method of claim 1, further comprising:
producing a report of the retrieved relevant subset of diagnosis classifications.

5. The method of claim 1, further comprising:
transmitting the retrieved relevant subset of diagnosis classifications to a remote user.

6. A method of improving the accuracy of a medical diagnosis of a patient by a healthcare provider, where the healthcare provider has examined the patient and identified at least one patient condition, the method comprising the steps of:
compiling a dataset of diagnosis classifications, the dataset derived from a plurality of industry standard databases;
receiving patient condition information, the patient condition information comprising a descriptive term;
comparing the descriptive term of the patient condition information with the dataset of diagnosis classifications, and deriving a relevant subset of diagnosis classifications;
displaying the relevant subset of diagnosis classifications for the healthcare provider to review and select an item from the relevant subset of diagnosis classifications for entry into a patient medical record.

7. The method of claim 6, further comprising:
receiving facility information about a healthcare facility where the patient is located;
comparing the facility information and the descriptive term of the patient condition information with the dataset of diagnosis classifications to derive the relevant subset of diagnosis classifications.

8. A system for assisting a healthcare provider to prepare a medical patient diagnosis report, the system comprising:
a database providing a plurality of medical condition classifications, the database indexed to associate at least one diagnosis criteria with a medical condition classification;
a database management engine communicating with the database and responding to queries containing patient diagnosis condition information to retrieve an associated medical condition classification, utilizing associations within the indexed database; and
a display screen displaying the retrieved associated medical condition classification.

9. The system of claim 8, where the retrieved associated medical condition classification is a plurality of medical condition classifications.

10. The system of claim 8, where the database is indexed by a facility location type.

11. The system of claim 8, where the database is indexed by a facility level of care.
